(19) United States
(12) Patent Application Publication
(10) Pub. No.: US 2022/0363727 A1
(43) Pub. Date: Nov. 17, 2022

(54) ANTIBACTERIAL PEPTIDE DERIVED FROM ERYTHROCULTER ILISHAEFORMIS AND USE THEREOF

(71) Applicant: SOOCHOW UNIVERSITY, Suzhou (CN)

(72) Inventors: Lin WEI, Suzhou (CN); Yue CHEN, Suzhou (CN); Honglan CHENG, Suzhou (CN)

(21) Appl. No.: 17/628,552

(22) PCT Filed: Nov. 26, 2020
(86) PCT No.: PCT/CN2020/131654

§ 371 Date: Jan. 19, 2022

(30) Foreign Application Priority Data
Nov. 19, 2020 (CN) 202011302943.1

(51) Int. Cl. C07K 14/46; A61K 31/43; A61K 38/17; A61P 31/04

(52) U.S. Cl. CPC C07K 14/461; A61K 31/43; A61K 38/1706; A61P 31/04

(57) ABSTRACT

The present invention relates to an antibacterial peptide derived from Erythroculter ilishaeformis and use thereof. The amino acid sequence of the antibacterial peptide of the present invention includes an amino acid sequence as shown in SEQ ID NO: 1. The present invention further discloses use of the antibacterial peptide in the preparation of an anti-aquatic-bacterium drug for aquaculture animals. The present invention provides a novel antibacterial peptide, discloses a function of the novel antibacterial peptide for resisting aquatic bacteria, and provides candidate molecules for preventing and treating bacterial diseases in the culture process of Erythroculter ilishaeformis.

### FIELD OF THE INVENTION

The present invention relates to an antibacterial peptide, and more particularly to an antibacterial peptide derived from Erythroculter ilishaeformis and use thereof.

### DESCRIPTION OF THE RELATED ART

Topmouthculter (Erythroculter ilishaeformis) belongs to the genus Cypriniformes, Cyprinidae, Barbinae, Erythroculter. Erythroculter ilishaeformis has the characteristics of fast growth and large individual size, and has the fastest growth rate and the largest individual size among the fishes of the genus Erythroculter. Due to the high nutritional value in muscle of Erythroculter ilishaeformis, the demand for Erythroculter ilishaeformis becomes increasingly higher. At present, large-scale Erythroculter ilishaeformis breeding bases have been developed in the Taihu Lake Basin.

However, high-density farming also brings a lot of farming diseases to fish, for example, bacterial infectious diseases. At present, for the prevention and treatment of bacterial infectious diseases in farmed fish, the main method is chemical drug therapy, supplemented by biological control. Although “drug treatment” has quick results, a large amount of drug residues is caused, which affects the safety of consumption of Erythroculter ilishaeformis, and also causes environmental pollution, thereby restricting the development of the aquaculture industry. In addition, repeated use of “drug therapy”, especially antibiotics, will aggravate the occurrence of bacterial resistance, imposing huge potential hazards.

Therefore, it is necessary to explore the composition characteristics of the immune system of Erythroculter ilishaeformis and the structure, function and mechanism of antibacterial peptides, to provide candidate molecules for the prevention and control of bacterial diseases in the Erythroculter ilishaeformis breeding process.

### SUMMARY OF THE INVENTION

In order to solve the above technical problems, an object of the present invention is to provide an antibacterial peptide derived from Erythroculter ilishaeformis and thereof. The present invention provides a novel antibacterial peptide, discloses a function of the novel antibacterial peptide for resisting aquatic bacteria, and provides candidate molecules for preventing and treating bacterial diseases in the culture process of Erythroculter ilishaeformis.

The first object of the present invention is to provide an antibacterial peptide, where the amino acid sequence of the antibacterial peptide of the present invention includes an amino acid sequence as shown in SEQ ID NO:1. The amino acid sequence as shown in SEQ ID NO:1 is a mature peptide sequence of the antibacterial peptide.

Preferably, a nucleotide sequence encoding the antibacterial peptide includes a nucleotide sequence as shown in SEQ ID NO:2.

Preferably, the amino acid sequence of the antibacterial peptide includes an amino acid sequence as shown in SEQ ID NO:3. The amino acid sequence as shown in SEQ ID NO:3 is a precursor of the antibacterial peptide. The precursor belongs to the LEAP-2 family of antibacterial peptides. The precursor includes a signal peptide sequence (SEQ ID NO:5), a leader peptide sequence (SEQ ID NO:6) and a mature peptide sequence (SEQ ID NO:1).

Preferably, a nucleotide sequence encoding the antibacterial peptide includes a nucleotide sequence as shown in SEQ ID NO:4. The nucleotide sequence as shown in SEQ ID NO: 4 is a full-length cDNA sequence of the antibacterial peptide, which contains 522 bases and encodes a precursor containing 92 amino acids. The full-length cDNA sequence further includes a polyadenylation site (aataa).

The second object of the present invention is to disclose use of the antibacterial peptide in the preparation of an anti-aquatic-bacterium drug for aquaculture animals.

Preferably, the aquaculture animals include Erythroculter ilishaeformis.

Preferably, the aquatic bacteria is selected from the group consisting of Aeromonas sobria, Aeromonas hydrophila, Vibrio harveyi, Vibrio parahaemolyticus, Vibrio anguillarum, Vibrio vulnificus, Vibrio splendidus, Vibrio cholerae and any combination thereof.

Preferably, the anti-aquatic-bacterium drug further includes ampicillin.

Preferably, the mass ratio of the antibacterial peptide to ampicillin in the anti-aquatic-bacterium drug is 1:1-4:1.

By means of the above technical solution, the present invention has the following advantages.

The present invention develops an antibacterial peptide derived from the immune system of Erythroculter ilishaeformis, identifies its structure, function and mechanism against infection by aquatic bacteria, and provides effective candidate molecules for preventing and treating bacterial diseases in the culture process of Erythroculter ilishaeformis.

The antibacterial peptide of the present invention has a small molecular weight, and can be synthesized chemically, or obtained through large-scale fermentation by constructing its encoding gene into a prokaryotic or eukaryotic expression vector. The antibacterial peptide of the present invention has a direct killing effect on aquaculture pathogenic bacteria, including drug-resistant bacteria, has a fast sterilization speed and is lethal.

The above description is only a summary of the technical solutions of the present invention. To make the technical means of the present invention clearer and implementable in accordance with the disclosure of the specification, the preferred embodiments of the present invention will be described in detail with reference to the accompanying drawings.

### BRIEF DESCRIPTION OF THE DRAWINGS

FIG. 1 shows the result of Sephadex G-50 gel chromatography and the result of reversed-phase liquid chromatography of antibacterial activity peak after Sephadex G-50 gel chromatography in the separation and purification process of the antibacterial peptide LEAP-2 from Erythroculter ilishaeformis;

FIG. 2 is a diagram showing comparison and analysis of the precursor of LEAP-2 from Erythroculter ilishaeformis and precursors of several LEAP-2 derived from cyprinids; and

FIG. 3 shows the SEM observation results of the effects of PBS and LEAP-2 from Erythroculter ilishaeformis on the cell membrane morphology of Aeromonas hydrophila.

### DETAILED DESCRIPTION OF THE PREFERRED EMBODIMENTS

The specific embodiments of the present invention will be described in further detail with reference to examples. The following examples are intended to illustrate the present invention, instead of limiting the scope of the present invention.

### EXAMPLE 1

### Separation and Purification of Antibacterial Peptide from Erythroculter ilishaeformis

Fresh Erythroculter ilishaeformis were captured from Taihu Lake, with a body length of 15-20 cm. Erythroculter ilishaeformis were kept fresh on ice, dissected to collect immune-related lymphoid tissues and organs such as head kidney, spleen, liver, etc. 20 mM PBS (1 mL/100 mg tissue) containing 1% (v/v) protease inhibitor was added and homogenized on ice, followed by addition of liquid nitrogen. The mixture was ground three times, centrifuged at 5000 g for 30 min. The supernatant was collected and lyophilized at low temperature for later use. The next step is to perform Sephadex G-50 gel filtration chromatography. The lyophilized powder was dissolved in 0.1 M Na₂HPO₄—NaH₂PO₄ phosphate buffer of pH 6.0, and the sample was loaded on a well-balanced Sephadex G-50 (Superfine, GE healthcare) gel filtration column (100 cm×2.6 cm), eluted with the same buffer and collected with an auto parts collector at a flow rate of 3 mL/tube/10 minutes. The light absorption at 280 nm of the collected solution was monitored. The peaks were combined and lyophilized for antibacterial activity test. The antibacterial activity peak was subjected to reversed-phase high performance liquid chromatography (RP-HPLC). Gradient elution was carried out using an elution system composed of water (containing 0.1% trifluoroacetic acid): acetonitrile (containing 0.1% trifluoroacetic acid) at 0.7 mL/min. The light absorption at 280 nm was monitored by ultraviolet. The peaks were collected, concentrated, lyophilized, and tested for antibacterial activity. The purified antibacterial activity peak was analyzed by matrix-assisted laser analysis and ionization time-of-flight mass spectrometry (MALDI-TOF-MS) using an UltraFlex I mass spectrometer (Bruker Daltonics), to analyze the purity and molecular weight of the purified antibacterial peptide. The N-terminal sequencing of the purified antibacterial peptide was performed by Edman degradation method (model 491, ABI, USA).

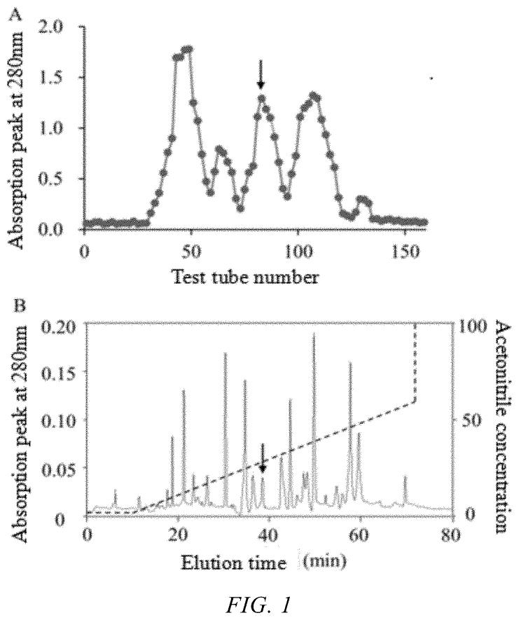

The results are as shown in FIG. 1. After being concentrated and subjected to Sephadex G-50 gel chromatography, the supernatant of the head kidney homogenate of Erythroculter ilishaeformis was divided into 5 eluted peaks. After antibacterial test, the third eluted peak (indicated by an arrow) has antibacterial activity (FIG. 1A). The third eluted peak of Sephadex G-50 gel chromatography was further divided into about 20 eluted peaks by reversed-phase liquid chromatography. After antibacterial test, the 10th eluted peak (indicated by an arrow) has antibacterial activity (FIG. 1B). Next, the molecular weight of the antibacterial peptide LEAP-2 contained in the 10th eluted peak purified in FIG. 1B was identified by mass spectrometry. The results are as shown in Table 1. The molecular weight was calculated to be 4652.17 Daltons. Further, sequencing was carried out by Edman degradation method. The obtained amino acid sequence of the N-terminus of the polypeptide is MTPLWRIMLLFKPHALCQNNY.

TABLE 1
Physical and chemical property parameters of
LEAP-2 from Erythroculter ilishaeformis

| | Net | Intramolecular | | Theoretical | Measured |
|---|---|---|---|---|---|
| Amino | positive | disulfide | | molecular | molecular |
| acid | charge | bond | pI^{a} | weight M_{W}^{b} | weight M_{W} |
| 41 | +3 | 2 | 8.91 | 4652.56 | 4652.17 |

^{a}isoelectric point;
^{b}molecular weight (Da)

### EXAMPLE 2

### Gene Cloning and Sequence Analysis of Antibacterial Peptide from Erythroculter ilishaeformis

Total RNA of the head kidney of Erythroculter ilishaeformis was extracted using Trizol, and a cDNA library of the head kidney of Erythroculter ilishaeformis was constructed with SMART™ cDNA Library Construction Kit from Clontech. First, a 5′ terminal fragment encoding LEAP -2 from Erythroculter ilishaeformis was cloned using a 5′ PCR primer (5′-AAGCAGTGGTATCAACGCAGAGT-3′, provided by the cDNA Library Construction Kit) and a degenerate primer S1 (5′-A(A/G)CAT(G/A/T)ATIC(G/T)CCA(A/G/C/T)A(A/G) (A/G/C/T)GG-3′, designed according to the amino acid sequence of Edman degradation sequencing). Then, the full-length cDNA sequence encoding LEAP-2 from Erythroculter ilishaeformis was cloned using a forward primer (5′-ATGCAGACCCACCCCAACAG-3′, a primer designed based on the cloned 5′ terminal fragment) and a 3′ PCR primer (5′-ATTCTAGAGGCCGAGGCGGCCGACATG-3′, provided by the cDNA Library Construction Kit).

As shown in SEQ ID NO:3-4, The full-length cDNA sequence encoding LEAP-2 from Erythroculter ilishaeformis contains 522 bases and encodes a precursor containing 92 amino acids. As proved by BLAST comparison, the precursor belongs to the LEAP-2 family of antibacterial peptides. The precursor includes a signal peptide sequence (SEQ ID NO:5), a leader peptide sequence (SEQ ID NO:6) and a mature peptide sequence (SEQ ID NO:1). The mature peptide sequence encoded by the cDNA contains 41 amino acids, and the N-terminus sequence of the encoded mature peptide is exactly the same as the N-terminus sequence obtained by Edman sequencing. The theoretical molecular weight of the encoded mature peptide is 4652.56 Daltons, the net positive charge is +3, and the theoretical isoelectric point is 8.91 (Table 1).

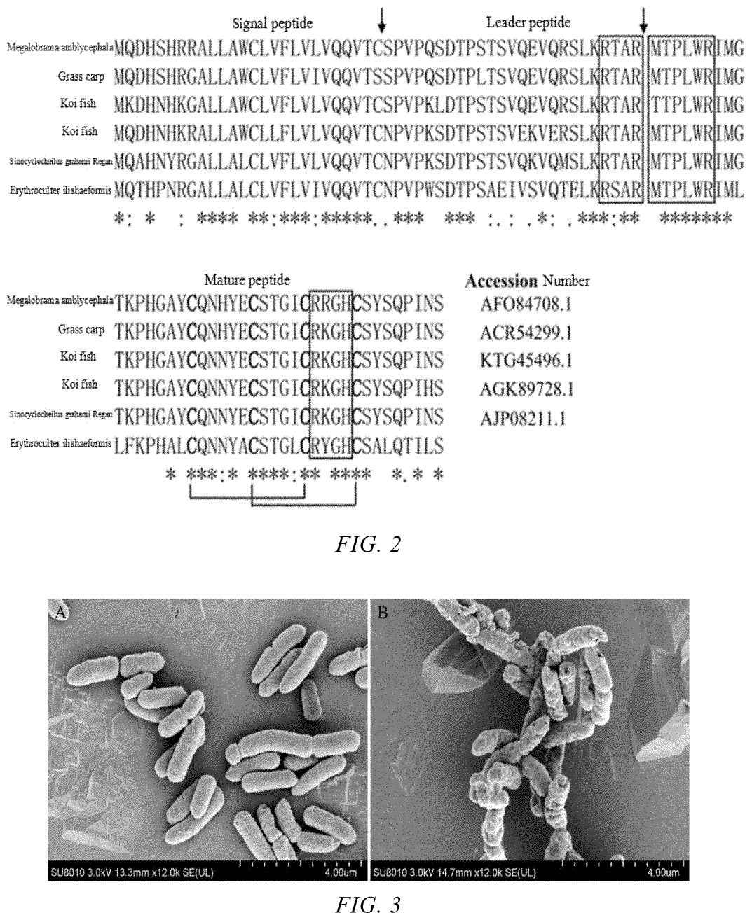

Then the precursor of LEAP-2 from Erythroculter ilishaeformis was compared with precursors of some LEAP-2 of cyprinids, as shown in FIG. 2. FIG. 2 shows comparison and analysis of the precursor of LEAP-2 from Erythroculter ilishaeformis and precursors of several LEAP-2 derived from cyprinids. In FIG. 2, “*” represent consistent sites, “:” represent conservative sites, “.” represent relatively conservative sites, the arrows indicate cleavage sites of the predicted signal peptide and the mature peptide, and the boxes represent the conservative sequences of “RXXR”, “MTPLWR” and “RXGH” respectively. The four conservative cysteines are marked in bold, and the line connecting the two cysteines is an intramolecular disulfide bond. It can be seen from FIG. 2 that LEAP-2 from Erythroculter ilishaeformis and LEAP-2 from these known cyprinids have the following common features: (1) There is a conservative “RXXR” (amino acid residue XX=TA or SA) amino acid sequence before the cleavage site of the mature peptide, which distinguishes the mature peptide from the leader peptide. (2) There is a conservative “MTPLWR” amino acid sequence at the N-terminus of the mature peptide. (3) There is a conservative “RXGH” (amino acid residue X=T or S) amino acid sequence at the C-terminus of the mature peptide. (4) LEAP-2 from these cyprinids all have 4 conservative Cys residues. According to the literature, the four Cys residues will form two intramolecular disulfide bonds, and are connected in the following manner: Cys1-Cys2 and Cys2-Cys4.

### EXAMPLE 3

### Analysis of Antibacterial Activity of Leap-2 from Erythroculter ilishaeformis

The antibacterial activity of LEAP-2 from Erythroculter ilishaeformis (having the amino acid sequence as shown in SEQ ID NO: 1) against fish pathogens was tested by a two-fold dilution method. LEAP-2 dilutions of a series of two-fold dilution gradient concentrations were prepared in a 96-well plate with a volume of 50 μL/well, and PBS and ampicillin were used as negative and positive controls, respectively. Aeromonas sobria, Aeromonas hydrophila, Vibrio harveyi, Vibrio parahaemolyticus, Vibrio anguillarum, Vibrio vulnificus, Vibrio splendidus and Vibrio cholerae were cultured in a nutrient broth medium to the logarithmic phase, and then diluted with fresh nutrient broth medium to 10⁵ CFU/mL. Then, 50 μL of diluted bacterial solution was added to each well of the 96-well plate containing the prepared two-fold dilution of LEAP-2 from Erythroculter ilishaeformis. After culturing at 37° C. for 18 hours, the minimal inhibitory concentration (MIC) was measured.

The results are shown in Table 2. The 8 strains tested were all sensitive to the antibacterial peptide LEAP-2 from Erythroculter ilishaeformis, and the concentration range of the MIC value was 18.75-150 μg/mL. Among the tested strains, Aeromonas sobria, Aeromonas hydrophila, Vibrio anguillarum, Vibrio vulnificus, Vibrio splendidus and Vibrio cholerae are resistant to the positive control ampicillin, and the MIC value is higher than 200 μg/mL. However, these strains resistant to ampicillin are all sensitive to LEAP-2 from Erythroculter ilishaeformis.

TABLE 2
Antibacterial activity of LEAP-2 from Erythroculter ilishaeformis

| | MIC (μg/mL)^{a} | | |
|---|---|---|---|
| Microorganism | | LEAP-2 | Ampicillin |

| | | | |
|---|---|---|---|
| Aeromonas sobria | 75 | (16.1 μM) | >200 |
| Aeromonas hydrophila | 18.75 | (4.0 μM) | >200 |
| Vibrio harveyi | 75 | (16.1 μM) | 37.5 |
| Vibrio parahaemolyticus | 75 | (16.1 μM) | 18.75 |
| Vibrio anguillarum | 150 | (32.2 μM) | >200 |
| Vibrio vulnificus | 37.5 | (8.1 μM) | >200 |
| Vibrio splendidus | 150 | (32.2 μM) | >200 |
| Vibrio cholerae | 37.5 | (8.1 μM) | >200 |

^{a}MIC: minimum inhibitory concentration. The concentration in the above table is the average of three independent experiments.

### EXAMPLE 4

### Kinetic Analysis of Antibacterial Action of Leap-2 from Erythroculter ilishaeformis

Aeromonas hydrophila (penicillin-resistant strain) was cultured to the logarithmic phase and diluted with fresh nutrient broth medium to 10⁵ CFU/mL. LEAP-2 from Erythroculter ilishaeformis (5×MIC, 93.75 μg/mL), ampicillin (1 mg/mL) and an equal volume of PBS were added to the diluted bacterial solution, and incubated at 37° C. for 0, 10, 20, 30, 45, 60, 90, 120, 180 minutes, respectively. At each time point, 50 μL of bacterial solution was taken and diluted 1000 times with nutrient broth medium. Then 50 μL of the dilution was spread on a nutrient broth agarose plate. After culturing at 37° C. for 12 hours, the CFU was calculated.

The results are shown in Table 3. Compared with the PBS treatment group, LEAP-2 from Erythroculter ilishaeformis inhibited the growth of Aeromonas hydrophila very well. LEAP-2 from Erythroculter ilishaeformis (5×MIC, 93.75 μ/mL, i.e., 20.2 μM) killed all bacteria within 60 minutes, and with the elapse of time, Aeromonas hydrophila did not grow again, indicating that its bactericidal effect on Aeromonas hydrophila is lethal. However, after treatment with ampicillin (1 mg/mL, 2862.0 μM) for 180 minutes, the growth of Aeromonas hydrophila was not completely inhibited. On the contrary, after 180 minutes of incubation, the CFU of Aeromonas hydrophila increased drastically from 6.7×10⁴ CFUs/mL to 9.6×10⁵ CFUs/mL.

TABLE 3
Kinetic analysis of antibacterial action of LEAP-2 from Erythroculter ilishaeformis on Aeromonas hydrophila
Number of bacteria co-cultured with different samples at different times (×10³, CFUs/mL)^{a}

| | Time (min) | | | | | | | | |
|---|---|---|---|---|---|---|---|---|---|
| Sample | 0 | 10 | 20 | 30 | 45 | 60 | 90 | 120 | 180 |
| LEAP-2 | 65 ± 5.3 | 59 ± 7.4 | 40 ± 8.4 | 28 ± 5.9 | 11 ± 3.5 | 0 | 0 | 0 | 0 |
| Ampicillin | 67 ± 7.5 | 69 ± 8.1 | 70 ± 7.2 | 72 ± 6.9 | 85 ± 6.9 | 123 ± 10.7 | 197 ± 12.6 | 378 ± 41.5 | 961 ± 87.3 |
| PBS | 62 ± 7.9 | 63 ± 9.2 | 77 ± 8.3 | 84 ± 11.6 | 149 ± 18.2 | 218 ± 30.1 | 324.3 ± 39.2 | 542 ± 75.4 | 1527 ± 271.7 |

### EXAMPLE 5

### Effect of Leap-2 from Erythroculter ilishaeformis on the Cell Membrane Morphology of the Bacteria

Aeromonas hydrophila (penicillin-resistant strain) was cultured to the logarithmic phase, washed with PBS and resuspended to 5×10⁶ CFU/mL. LEAP-2 from Erythroculter ilishaeformis (5×MIC, 93.75 μg/mL) was added to the bacterial suspension, incubated at 37° C. for 30 minutes, and centrifuged at 1,000 g for 10 minutes. The bacterial cells were fixed with 2.5% glutaraldehyde. A sample was prepared according to the standard procedure of the scanning electron microscope, and observed with Hitachi SU8010.

The result is as shown in FIG. 3. The Aeromonas hydrophila treated with PBS (negative control) showed a regular, smooth and intact surface (FIG. 3A), while the membrane surface of Aeromonas hydrophila in the treatment group using LEAP-2 from Erythroculter ilishaeformis significantly changed, where many protruding vesicles or even perforations were formed on the surface, and some bacteria were covered with irregular bacterial fragments (FIG. 3B).

### EXAMPLE 6

### Leap-2 from Erythroculter ilishaeformis and Ampicillin have a Synergistic Effect

It is evaluated whether LEAP-2 from Erythroculter ilishaeformis and ampicillin have a synergistic effect by using a checkerboard test method. Two-fold dilutions of LEAP-2 from Erythroculter ilishaeformis and Ampicillin were added in a 96-well plate at the same time. The concentrations of the two dilutions were both 200 μg/mL, 100 μg/mL, 50 μg/mL, 25 μg/mL, 12.5 μg/mL, 6.25 μg/mL, 3.125 μg/mL, 1.5625 μg/mL, 0.78125 μg/mL, 0.390625 μg/mL, 0.1953125 μg/mL or 0.09765625 μg/mL. 50 μL was added to each well, and uniformly mixed. V. harveyi and V. parahaemolyticus were cultured to the logarithmic phase, diluted to 10⁵ CFU/mL with fresh nutrient broth medium, and then added to the 96-well plate containing LEAP-2 and ampicillin. 100 μL of bacterial solution was added to each well. After culturing at 37° C. for 18 hours, the light absorption value at OD600 nm was measured, the growth of bacteria was tested, and the fractional inhibitory concentration index (FICI) between LEAP-2 from Erythroculter ilishaeformis and ampicillin was calculated.

The results are shown in Table 4. In the antibacterial test of V. harveyi, the FICI value is 0.375 when LEAP-2 from Erythroculter ilishaeformis and ampicillin are used in combination, indicating that they have a synergistic effect. In the antibacterial test of Vibrio parahaemolyticus (V. parahaemolyticus), the FICI value is 0.5 when LEAP-2 from Erythroculter ilishaeformis and ampicillin are used in combination, also indicating that they have a synergistic effect. Therefore, it indicates that LEAP-2 from Erythroculter ilishaeformis and ampicillin have a synergistic effect.

TABLE 4
LEAP-2 from Erythroculter ilishaeformis
and ampicillin having a synergistic effect

| | | MIC (μg/mL) | | | |
|---|---|---|---|---|---|
| | Pharmaceutical | Used | Used in | | |
| Microorganism | composition | alone | combination | FIC | FICI^{a} |
| Vibrio | Ampicillin | 37.5 | 4.69 | 0.125 | 0.375 |
| harveyi | LEAP-2 | 75 | 18.75 | 0.25 | |
| Vibrio | Ampicillin | 18.75 | 4.69 | 0.25 | 0.5 |
| parahaemolyticus | LEAP-2 | 75 | 18.75 | 0.25 | |

^{a}The FICI was calculated using the following formula: FICI = FIC (ampicillin) + FIC (LEAP-2), where FIC of ampicillin = MIC of ampicillin used in combination with LEAP-2/MIC of ampicillin used alone, and FIC of LEAP-2 = MIC of LEAP-2 used in combination with ampicillin/MIC of LEAP-2 used alone. When FICI ≤ 0.5, it means that ampicillin and LEAP-2 have a synergistic effect; when 0.5 ≤ FICI ≤ 1.0, it means that ampicillin and LEAP-2 have an additive effect; when 1.0 ≤ FICI ≤ 4.0, it means that ampicillin and LEAP-2 do not interact with each other; when FICI > 4.0, it means that ampicillin and LEAP-2 have an antagonistic effect. The concentration in the above table is the average of three independent experiments.

While preferred embodiments of the present invention have been described above, the present invention is not limited thereto. It should be appreciated that some improvements and variations can be made by those skilled in the art without departing from the technical principles of the present invention, which are also contemplated to be within the scope of the present invention.

1. An antibacterial peptide, wherein an amino acid sequence of the antibacterial peptide comprises an amino acid sequence as shown in SEQ ID NO:1.

2. The antibacterial peptide according to claim 1, wherein a nucleotide sequence encoding the antibacterial peptide comprises a nucleotide sequence as shown in SEQ ID NO:2.

3. The antibacterial peptide according to claim 1, wherein the amino acid sequence of the antibacterial peptide comprises an amino acid sequence as shown in SEQ ID NO:3.

4. The antibacterial peptide according to claim 3, wherein the nucleotide sequence encoding the antibacterial peptide comprises a nucleotide sequence as shown in SEQ ID NO:4.

5. Use of the antibacterial peptide according to claim 1 in the preparation of an anti-aquatic-bacterium drug for aquaculture animals.

6. The use according to claim 5, wherein the aquaculture animals comprise Erythroculter ilishaeformis.

7. The use according to claim 5, wherein the aquatic bacteria is selected from the group consisting of Aeromonas sobria, Aeromonas hydrophila, Vibrio harveyi, Vibrio parahaemolyticus, Vibrio anguillarum, Vibrio vulnificus, Vibrio splendidus, Vibrio cholerae and any combination thereof.

8. The use according to claim 5, wherein the anti-aquatic-bacterium drug further comprises ampicillin.

9. The use according to claim 8, wherein the mass ratio of the antibacterial peptide to ampicillin in the anti-aquatic-bacterium drug is 1:1-4:1


SEQUENCE LISTING (6 sequences)

SEQ ID NO: 1 - protein, 41 aa, Artificial sequence
  Antibacterial peptide
MTPLWRIMLLFKPHALCQNNYACSTGLCRYGHCSALQTILS

SEQ ID NO: 2 - DNA, 123 bp, Artificial sequence
  Antibacterial peptide
atgacccccttatggcggatcatgttactctttaaacctcacgccctttgccagaacaattatgcttgctcaacaggactgtgcaggtatggccactgttccgctctgcaaacaatactatct

SEQ ID NO: 3 - protein, 92 aa, Artificial sequence
  Antibacterial peptide
MQTHPNRGALLALCLVFLVIVQQVTCNPVPWSDTPSAEIVSVQTELKRSARMTPLWRIMLLFKPHALCQNNYACSTGLCRYGHCSALQTILS

SEQ ID NO: 4 - DNA, 522 bp, Artificial sequence
  Antibacterial peptide
atgcagacccaccccaacagaggtgctcttctagccctgtgcctggtgtttttggtgatcgtccagcaggtaacctgcaacccagtgccttggtcggacacaccatcagccgaaatcgtgtcagttcaaaccgaactgaagagatctgctaggatgacccccttatggcggatcatgttactctttaaacctcacgccctttgccagaacaattatgcttgctcaacaggactgtgcaggtatggccactgttccgctctgcaaacaatactatcttaaaacgaagactccgtctttgctctctgatctgacgtttttacatcatgcatgacaagagcttaattcagtgaacaacaggggcaactgaatgtgcaatgcagttaataatcagttagacatatagtcaaggctagggctgactcctctaaagccacagaggaggcaatatgtaatcttcagagtattgggcttatcataaggtacataacgtgtactcttttgtgagtgtacaaaaaaaaaaaa

SEQ ID NO: 5 - protein, 28 aa, Artificial sequence
  Signal peptide
MQTHPNRGALLALCLVFLVIVQQVTCNP

SEQ ID NO: 6 - protein, 23 aa, Artificial sequence
  Leader peptide
VPWSDTPSAEIVSVQTELKRSAR